(19) United States
(12) Patent Application Publication
(10) Pub. No.: US 2014/0121429 A1
(43) Pub. Date: May 1, 2014

(54) PROPYLENE PRODUCTION PROCESS WITH HEAVIES RECYCLE

(71) Applicant: LYONDELL CHEMICAL TECHNOLOGY, L.P., Houston, TX (US)

(72) Inventors: Steven T. Coleman, Humble, TX (US); Gary A. Sawyer, Media, PA (US); Thomas S. Zak, West Chester, PA (US)

(73) Assignee: Lyondell Chemical Technology, L.P., Houston, TX (US)

(21) Appl. No.: 13/663,690

(22) Filed: Oct. 30, 2012

(51) Int. Cl. C07C 4/06

(52) U.S. Cl. 585/315

(57) ABSTRACT

Processes for forming propylene are described herein. The processes generally include reacting a metathesis feed stream including n-butene with ethylene in the presence of a metathesis catalyst via a metathesis reaction to form a metathesis product stream including propylene, ethylene, butene and C₅₊ olefins; separating the propylene from the ethylene, butene and C₅₊ olefins in the metathesis product stream; and recycling at least a portion of the C₅₊ olefins to the metathesis reaction.

### CROSS-REFERENCE TO RELATED APPLICATIONS

Not applicable.

### STATEMENT REGARDING FEDERALLY SPONSORED RESEARCH OR DEVELOPMENT

Not applicable.

### FIELD OF THE INVENTION

The present invention generally relates to propylene production processes. More particularly, the present invention relates to propylene production processes including recycle of C₅₊ olefins.

### BACKGROUND

This section introduces information from the art that may be related to or provide context for some aspects of the techniques described herein and/or claimed below. This information is background facilitating a better understanding of that which is disclosed herein. This is a discussion of “related” art. That such art is related in no way implies that it is also “prior” art. The related art may or may not be prior art. The discussion is to be read in this light, and not as admissions of prior art.

Propylene can be produced by the metathesis reaction of linear butene (n-butene) with ethylene. Such processes may produce C₅₊ olefins, which are often utilized for gasoline blending (i.e., C₅₊ olefinic gasoline). At a given temperature, one way to increase the production ratio of propylene to C₅₊ olefinic gasoline is by increasing the ethylene to butene ratio at the reactor inlet. However, such increase requires an increased ethylene recycle.

The present invention is directed to resolving, or at least reducing, one or all of the problems mentioned above,

### SUMMARY

Various embodiments of the present invention include processes for forming propylene. The processes generally include reacting a metathesis feed stream including n-butene with ethylene in the presence of a metathesis catalyst via a metathesis reaction to form a metathesis product stream including propylene, ethylene, butene and C₅₊ olefins; separating the propylene from the ethylene, butene and C₅₊ olefins in the metathesis product stream; and recycling at least a portion of the C₅₊ olefins to the metathesis reaction.

One or more embodiments include the process of the preceding paragraph and further include reacting the metathesis feed stream with ethylene in the presence of the metathesis catalyst and an isomerization catalyst to form the metathesis product stream.

One or more embodiments include the process of any preceding paragraph, wherein ethylene is introduced to the metathesis reaction at a rate sufficient to provide an ethylene:C₄₊ ratio contacting the metathesis catalyst of from 0.3:1 to 3:1.

One or more embodiments include the process of any preceding paragraph, wherein at least 5% of the C₅₊ olefins are recycled to the metathesis reaction.

One or more embodiments include the process of any preceding paragraph, wherein the metathesis catalyst includes a transition metal oxide.

One or more embodiments include the process of any preceding paragraph, wherein the metathesis catalyst includes tungsten oxide.

One or more embodiments include the process of any preceding paragraph, wherein at least 95% of the C₅₊ olefins are recycled to the metathesis reaction.

One or more embodiments include a process for forming propylene including reacting a metathesis feed stream including n-butene with ethylene in the presence of a metathesis catalyst via a metathesis reaction to form a metathesis product stream including propylene, ethylene, butene, and C₅₊ olefins; separating at least a portion of the propylene from the metathesis product stream to form an overhead stream and a de-propenized bottoms stream including butene and C₅₊ olefins; separating at least a portion of the ethylene from either the metathesis product stream or the overhead stream to form an ethylene stream; recovering the propylene from the overhead stream; recycling at least a portion of the ethylene stream to the metathesis reaction; separating at least a portion of the butene from the de-propenized bottoms stream to form a butene stream and a de-butenized bottoms stream; recycling at least a portion of the butene stream to the metathesis reaction; and recycling at least a portion of the de-butenized bottoms stream, a portion of the de-propenized bottoms stream or at least a portion of the de-butenized bottoms stream and a portion of the de-propenized bottoms stream to the metathesis reaction.

One or more embodiments include the process of the preceding paragraph and further includes reacting the metathesis feed stream with ethylene in the presence of the metathesis catalyst and an isomerization catalyst to form the metathesis product stream.

One or more embodiments include the process of any preceding paragraph, wherein the isomerization catalyst includes magnesium oxide.

One or more embodiments include the process of any preceding paragraph, wherein ethylene is introduced to the metathesis reaction at a rate sufficient to provide an ethylene:C₄₊ ratio contacting the metathesis catalyst of from 0.3:1 to 3:1.

One or more embodiments include the process of any preceding paragraph, wherein at least 60% of the butene stream is recycled to the metathesis reactor.

One or more embodiments include the process of any preceding paragraph, wherein the process exhibits a process efficiency of at least 95%.

One or more embodiments include the process of any preceding paragraph, wherein the metathesis catalyst includes a transition metal oxide.

One or more embodiments include the process of any preceding paragraph, wherein the metathesis catalyst includes tungsten oxide.

One or more embodiments include the process of any preceding paragraph, wherein from 5% to 90% of the de-propenized bottoms stream is recycled to the metathesis reactor.

One or more embodiments include the process of any preceding paragraph, wherein at least 80% of the butene stream is recycled to the metathesis reactor.

One or more embodiments include the process of any preceding paragraph, wherein at least 95% of the butene stream is recycled to the metathesis reactor.

The above paragraphs present a simplified summary of the presently disclosed subject matter in order to provide a basic understanding of some aspects thereof. The summary is not an exhaustive overview, nor is it intended to identify key or critical elements to delineate the scope of the subject matter claimed below. Its sole purpose is to present some concepts in a simplified firm as a prelude to the more detailed description set forth below.

### BRIEF DESCRIPTION OF DRAWINGS

The claimed subject matter may be understood by reference to the following description taken in conjunction with the accompanying drawings, in which like reference numerals identify like elements, and in which:

FIG. 1 illustrates a simplified process flow diagram of a process for producing propylene.

FIG. 2 illustrates the consumption of ethylene and production of propylene and C₅₊ olefinic gasoline versus fraction of C₅₊ olefinic gasoline being recycled in a specific example.

FIG. 3 illustrates reactor throughput versus process efficiency in a specific example.

While the invention is susceptible to various modifications and alternative forms, the drawings illustrate specific embodiments herein described in detail by way of example. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION

Illustrative embodiments of the subject matter claimed below will now be disclosed. In the interest of clarity, not all features of an actual implementation are described in this specification. It will be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort, even if complex and time-consuming, would be a routine undertaking fir those of ordinary skill in the art having the benefit of this disclosure.

In the description below, unless otherwise specified, all compounds described herein may be substituted or unsubstituted and the listing of compounds includes derivatives thereof. Further, various ranges and/or numerical limitations may be expressly stated below. It should be recognized that unless stated otherwise, it is intended that endpoints are to be interchangeable. Further, any ranges include iterative ranges of like magnitude falling within the expressly stated ranges or limitations.

Embodiments described herein include processes for forming propylene. The processes generally include reacting a metathesis feed stream including n-butene with ethylene in the presence of a metathesis catalyst to form a metathesis product stream including propylene, ethylene, butene and C₅₊ olefins; separating the propylene from the ethylene, butene and C₅₊ olefins and recycling at least a portion of the C₅₊ olefins to the metathesis reaction.

In one or more specific embodiments, the metathesis feed stream may be formed by contacting a first feed stream including ethylene with a dimerization catalyst to form a dimerization product stream including n-butene. As used herein, the term “dimerization” refers to a chemical reaction in which two identical molecular entities react to form a single dimer. In the present embodiments, the identical molecular entities are generally ethylene, while the dimer is generally butene.

The dimerization catalyst may include catalyst known in the art to be capable of converting ethylene to linear C₄ olefins (i.e., n-butene) upon reaction. For example, dimerization catalysts may include homogenous catalyst compounds including nickel. Many catalysts containing nickel are known to dimerize ethylene to butene (e.g., U.S. Pat. No. 4,528,415, U.S. Pat. No. 3,513,218 and U.S. Pat. No. 3,452,115).

Alternatively, the dimerization catalyst may include an organoaluminum compound of the formula RₙAlX₃₋ₙ, wherein R is selected from alkyls, such as butyl, ethyl and methyl, X is selected from halogens, such as chlorine and n is 0, 1 or 2, for example.

Although the dimerization may be carried out in any reactor type, a fixed-bed reactor is a specific example. The dimerization may be carried out under moderate conditions, such as temperatures of from 20° C. to 400° C., or from 25° C. to 150° C. or from 30° C. to 55° C. and pressures of from 200 psig to 400 psig, or from 250 psig to 350 psig or from 265 psig to 315 psig, for example.

it is further contemplated that, depending upon the nature of the source of the metathesis feed stream, the metathesis feed stream may undergo separation in a butene fractionation system (such as the de-butenizer described in further detail below) prior to utilization as the metathesis feed stream.

As mentioned above, embodiments described herein include reacting the metathesis feed stream with ethylene in the presence of a metathesis catalyst to form a metathesis product stream (i.e., a metathesis reaction). As used herein, the term “metathesis” refers to an equilibrium reaction between two olefins where the double bond of each olefin is broken to form intermediate reactants. These intermediates recombine to form new olefin products. In one or more specific embodiments discussed herein, the two olefins include ethylene and butene and the new olefin product is propylene.

Also as discussed previously herein, n-butene is fed to the metathesis reaction via the metathesis feed stream. The ethylene may be fed to the reactor by any suitable method known to one skilled in the art. For example, the ethylene may be fed to the metathesis reaction via an inlet separate from an inlet utilized to feed the metathesis feed stream. Alternatively, the ethylene may be combined with the metathesis feed stream prior to the metathesis feed stream passing through such inlet.

The metathesis reaction includes contacting the butene with ethylene in the presence of a metathesis catalyst. Metathesis catalysts are well known in the art (see, e.g., U.S. Pat. No. 4,513,099 and U.S. Pat. No. 5,120,894). Generally, the metathesis catalyst includes a transition metal oxide, such as transition metal oxides of cobalt, molybdenum, rhenium, tungsten and combinations thereof, for example. In one or more specific embodiments, the metathesis catalyst includes tungsten oxide. The metathesis catalyst may be supported on a carrier, such as silica, alumina, titania, zirconia, zeolites, clays and mixtures thereof, for example. In one or more embodiments, the carrier is selected from silica, alumina and combinations thereof. The catalyst may be supported on a carrier by methods known in the art, such as adsorption, ion-exchange, impregnation or sublimation, for example. The metathesis catalyst may include from 1 wt. % to 30 wt. % or from 5 wt. % to 20 wt. % transition metal oxide, for example.

The metathesis reaction may further include contacting the butene with ethylene in the presence of an isomerization catalyst (either sequentially or simultaneously with the metathesis catalyst). The isomerization catalyst is generally adapted to convert 1-butene present in the metathesis feed stream to 2-butene for subsequent reaction to propylene. Isomerization catalysts may include zeolites, metal oxides (e.g., magnesium oxide, tungsten oxide, calcium oxide, barium oxide, lithium oxide and combinations thereof), mixed metal oxides (e.g., silica-alumina, zirconia-silica), acidic clays (see, e.g., U.S. Pat. No. 5,153,165; U.S. Pat. No. 4,992,613; U.S. Patent Publication 2004/0249229 and U.S. Patent Publication 2006/0084831) and combinations thereof, for example. In one or more specific embodiments, the catalyst is magnesium oxide. The magnesium oxide may have a surface area of at least 1 m²/g or at least 5 m²/g, for example.

The isomerization catalyst may be supported on a support material. Suitable support materials include silica, alumina, titania, silica-alumina and combinations thereof for example.

The metathesis reactions may occur at a pressure of from 150 psig to 600 psig, or from 200 psig to 500 psig, or from 240 to 450 psig, for example. The metathesis reaction may occur at a temperature of from 100° C. to 500° C., or from 200° C. to 400° C. or from 300° C. to 350° C., for example. The metathesis reaction may occur at a WHSV of from 3 hr⁻¹ to 200 hr⁻¹ or from 6 hr⁻¹ to 40 hr^{'1}, for example,

The contact time needed to obtain a desirable yield of metathesis reaction products depends upon several factors, such as the activity of the catalyst, temperature and pressure, for example. However, in one or more embodiments, the length of time during which the metathesis feed stream and the ethylene are contacted with the catalyst can vary from 0.1 s to 4 hours or from 0.5 s to 0.5 hours, for example. The metathesis reaction may be conducted batch-wise or continuously with fixed catalyst beds, slurried catalyst, fluidized beds, or by using any other conventional contacting techniques, for example.

The metathesis product stream generally includes ethylene, propylene, C₄ olefins, and C₅₊ olefins (including pentene and hexane, for example). Therefore, the process generally includes separating the components of the metathesis product stream. An example of a method of separation is shown in U.S. Pat. No. 7,214,841, which is hereby incorporated by reference, and such methods generally include separation within a fractionation system (although it is contemplated that alternative methods, such as separation via a membrane, may be utilized). As used herein, the term “fractionation” refers to processes for the separation of components based on the relative volatility and/or boiling point of the components. The fractionation processes may include those known in the art and the term “fractionation” can be used interchangeably with the terms “distillation” and “fractional distillation” herein.

The fractionation system generally includes a de-ethenizer, a de-propenizer and a de-butenizer. The de-ethenizer receives and separates the metathesis product stream including propylene, ethylene, butene, and C₅₊ olefins to form a recycle ethylene stream and a de-ethenizer bottoms stream. The recycle ethylene stream is composed primarily of the recovered ethylene and at least a portion of the recycle ethylene stream may be recycled back to the metathesis reaction. The de-ethenizer bottoms stream generally includes the propylene, butene and C₅₊ olefins.

The de-propenizer receives and separates the de-ethenizer product to form a propylene stream and a de-propenizer bottoms stream. The propylene stream is composed primarily of the propylene product. The de-propenizer bottoms stream generally includes the butene and C₅₊ olefins.

in one or more specific embodiments, at least a portion of the de-propenizer bottoms stream 130 may be recycled back to the metathesis reaction. For example, from 0% to 95%, or from 0% to 30%, or from 0% to 25% or from 5% to about 20% of the de-propenizer bottoms stream 130 (which may be referred to as a first portion of the de-propenizer bottoms stream) may be recycled to the metathesis reaction.

The de-butenizer receives and separates at least a portion of the de-propenizer bottoms stream 130 (which may be referred to as a second portion of the de-propenizer bottoms stream when a first portion is recycled to the metathesis reaction) to form a recycle butene stream and a de-butenizer bottoms stream. The recycle butene stream 128 is composed primarily of the recovered butene and the de-butenizer bottoms stream 130 generally includes the C₅₊ olefins (interchangeably referred to herein as “C₅₊ olefinic gasoline”). A C₄ overhead purge 129 may be employed in some embodiments.

In one or more specific embodiments, at least a portion of the de-butenizer bottoms stream is recycled back to the metathesis reaction. For example, from 60% to 100%, or at least 70%, or at least 80% or at least 90% or at least 95% of the de-butenizer bottoms stream may be recycled to the metathesis reaction. Any de-butenizer bottoms stream that is not recycled may be utilized as C₅₊ olefinic gasoline product (i.e., heavier olefin stream suitable for gasoline blending).

The molar ratio of ethylene to C₄₊ olefins contacting the metathesis catalyst may range from 0.1:1 to 3:1, or from 0.3:1 to 2:1 or from 1:1 to 2:1, for example.

The embodiments described herein (i.e., recycle of C₅₊ olefins to the metathesis reaction) can result in increased propylene production (compared to identical processes absent C₅₊ olefinic gasoline recycle) without a significant increase in ethylene consumption. Furthermore, embodiments described herein are capable of high process efficiencies (e.g., at least 85%, or at least 95% or at least 98%). As used herein, the term “process efficiency” is defined as (propylene production minus C₅₊ olefinic gasoline production minus ethylene feed) divided by net butene feed.

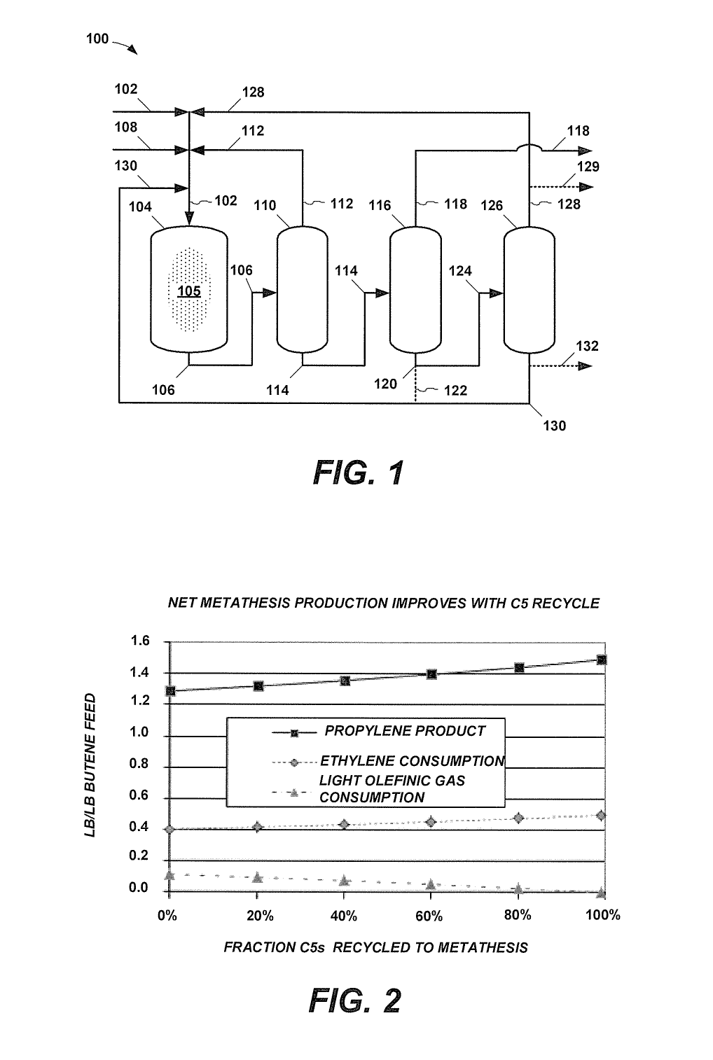

Referring now to FIG. 1, a simplified process flow diagram of a process 100 for producing propylene according to embodiments disclosed herein is illustrated. FIG. 1 illustrates a process 100 including introducing a metathesis feed stream 102 to a metathesis reactor 104 having metathesis catalyst 105 (and optional isomerization catalyst-not shown) disposed therein to form metathesis product stream 106 including propylene, ethylene, butene and C₅₊ olefins. FIG. 1 illustrates a specific embodiment wherein ethylene is mixed with the metathesis feed stream 102 via line 108; however, it is contemplated that the ethylene may contact the metathesis feed stream via processes known in the art.

The metathesis product stream 106 is passed to a de-etherizer 110 to separate at least a portion of the ethylene from the metathesis product stream 106 to form a recycle ethylene stream 112 and a de-ethenizer bottoms stream 114 including propylene and C₄₊ olefins. In the specific embodiment illustrated in FIG. 1, the recycle ethylene stream 112 is recycled to the metathesis reactor 104 via methods known in the art.

The de-ethenizer bottoms stream 114 is passed to a de-propenizer 116 to separate at least a portion of the propylene from the de-ethenizer bottoms stream 114 and form a propylene stream 118 and a de-propenizer bottoms stream 120 including C₄₊ olefins. Optionally, at least a portion (i.e., a first portion) of the de-propenizer bottoms stream 120 may be recycled to the metathesis reactor 104 through line 122 via known methods. The portion of the de-propenizer bottoms stream 120 that is not recycled (in some embodiments, all of the de-propenizer bottoms stream 120 and in other embodiments, a second portion) passes via line 124 to a de-butenizer 126.

The de-butenizer 126 separates at least a portion of the butene from the de-propenizer bottoms stream 124 to form a recycle butene stream 128 and a de-butenizer bottoms stream 130 including C₅₊ olefins. At least a portion of the recycle butene stream 128 and at least a portion of the de-butenizer bottoms stream 130 is recycled to the metathesis reactor 104. Optionally, a portion of the de-butenizer bottoms stream is withdrawn from the process 100 as a purge stream 132.

Those in the art having the benefit of this disclosure will recognize that there are a number of suitable separation techniques well known to the art that may be used to achieve this separation. Any such suitable technique may be used.

### EXAMPLES

To facilitate a better understanding of the present invention, the following examples of embodiments are given. In no way should the following examples be read to limit, or to define, the scope of the invention.

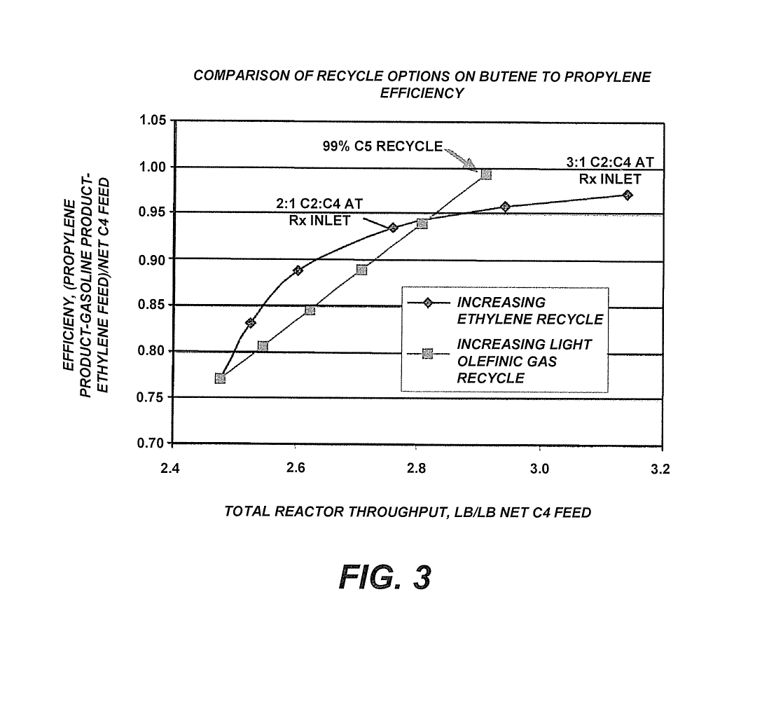

Material balances based on simultaneous equilibrium of metathesis and olefin isomerization reactions were prepared for various ethylene or C₅₊ olefinic gasoline recycle ratios based on the embodiments described herein. FIG. 2 shows the consumption of ethylene and production of propylene and C₅₊ olefinic gasoline versus fraction of C₅₊ olefinic gasoline being recycled. Reaction conditions of 650° F. and inlet 1:1 molar ethylene: (butenes plus pentenes) were constant with changing C₅₊ olefinic gasoline recycle. Comparing the options of increasing ethylene recycle versus C₅₊ olefinic gasoline recycle, FIG. 3 shows that increasing ethylene recycle has diminishing returns, while recycling the C₅₊ olefinic gasoline approaches nearly 100% butene efficiency with less total flow through the metathesis reactor at high efficiencies.

### CLOSING OF THE DETAILED DESCRIPTION

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present invention.

The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values.

The following patents and/or patent applications are hereby incorporated by reference for the purposes described above as if set forth verbatim herein:

U.S. Pat. No. 4,528,415, entitled “Ethylene Dimerization”, and issued Jul. 9, 1985, in the name of the inventor(s) Ronald D. Knudsen et al. and assigned on its face to Phillips Petroleum Co.;

U.S. Pat. No. 3,513,218, entitled “Olefin Dimerization”, and issued May 19, 1970, in the name of the inventor(s) Volkert Falkings et al. and assigned on its face to Solven Cheral;

U.S. Pat. No. 3,452,115, entitled “Process for Dimerization of Olefins”, and issued Jun. 24, 1969, in the name of the inventor(s) Wolfgang Schneider et al. and assigned on its face to B.F. Goodrich Co.;

U.S. Pat. No. 4,513,099, entitled “Olefin Metathesis and Catalyst”, and issued Apr. 23, 1985, in the name of the inventor(s) Simon G. Kukes et al. and assigned on its face to Phillips Petroleum Co.;

U.S. Pat. No. 5,120,894, entitled “Olefin Conversion Process”, and issued Jun. 9, 1992, in the name of the inventor(s) Michael W. McCauley et al. and assigned on its face to Lyondell Petrochemical Co.;

U.S. Pat. No. 5,153,165, entitled “Preparation of alkaline earth oxide catalysts”, and issued Oct. 6, 1992, in the name of the inventor(s) Richard E. Lowery et al. and assigned on its face to Phillips Petroleum Co.;

U.S. Pat. No. 4,992,613, entitled “Double-bond isomerization process using basic zeolite catalysts”, and issued Feb. 12, 1991, in the name of the inventor(s) Thomas F. Brownscombe et al. and assigned on its face to Shell Oil Co.;

U.S. Pat. No. 7,214,841, entitled “Processing C4 olefin streams for the maximum production of propylene”, and issued May 8, 2007, in the name of the inventor(s) Robert J. Gartside et a., and assigned on its face to ABB Lummus Global Inc.;

U.S. Patent Publication 2004/0249229, entitled “Isomerization of olefins with carboxylic acid”, and issued December 9, 2004, in the name of the inventor(s) Jeffery C. Gee et al. and assigned on its face to Chevron Phillips Chemical Co.;

U.S. Patent Publication 2006/0084831, entitled “Process for isomerization of alpha olefins to internal olefins”, and issued Apr. 20, 2006, in the name of the inventor(s) Jian Jian Zhang and assigned on its face to Hercules Inc.

To the extent any incorporated patent, patent application, or other reference conflicts with the present disclosure, the present disclosure controls.

This concludes the detailed description. The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

1. A process for forming propylene comprising:
reacting a metathesis feed stream comprising n-butene with ethylene in the presence of a metathesis catalyst via a metathesis reaction to form a metathesis product stream comprising propylene, ethylene, butene and C₅₊ olefins;
separating the propylene from the ethylene, butene and C₅₊ olefins in the metathesis product stream; and
recycling at least a portion of the C₅₊ olefins to the metathesis reaction to produce a C₄₊ stream comprising butene and C₅₊ olefins,
wherein ethylene is introduced to the metathesis reaction at a rate sufficient to provide an ethylene:C₄₊ stream molar ratio contacting the metathesis catalyst of from 0.3:1 to 3:1
wherein the process for forming propylene has a butene to propylene efficiency between 80 and 100%.

2. The process of claim 1 further comprising reacting the metathesis feed stream with ethylene in the presence of the metathesis catalyst and an isomerization catalyst to form the metathesis product stream.

3. The process of claim 1, wherein ethylene is introduced to the metathesis reaction at a rate sufficient to provide an ethylene:C₄₊ molar ratio contacting the metathesis catalyst of from 1:1 to 2:1.

4. (canceled)

5. The process of claim 1, wherein the metathesis catalyst comprises a transition metal oxide.

6. The process of claim 1, wherein the metathesis catalyst comprises tungsten oxide.

7. (canceled)

8. A process for forming propylene comprising:
reacting a metathesis C₄₊ feed stream comprising C₅₊ olefins and n-butene with ethylene in the presence of a metathesis catalyst via a metathesis reaction to form a metathesis product stream comprising propylene, ethylene, butene, and C₅₊ olefins,
wherein ethylene is introduced to the metathesis reaction at a rate sufficient to provide an ethylene:C₄₊ feed stream molar ratio from 0.3:1 to 3:1;
separating at least a portion of the propylene from the metathesis product stream to form an overhead stream and a de-propenized bottoms stream comprising butene and C₅₊ olefins;
separating at least a portion of the ethylene from either the metathesis product stream or the overhead stream to form an ethylene stream;
recovering the propylene from the overhead stream;
recycling at least a portion of the ethylene stream to the metathesis reaction;
separating at least a portion of the butene from the de-propenized bottoms stream to form a butene stream and a de-butenized bottoms stream;
recycling at least a portion of the butene stream to the metathesis reaction; and
recycling at least a portion of the de-butenized bottoms stream, a portion of the de-propenized bottoms stream or at least a portion of the de-butenized bottoms stream and a portion of the de-propenized bottoms stream to the metathesis reaction wherein the process for forming propylene has a butene to propylene efficiency between 80 and 100%.

9. The process of claim 8 further comprising reacting the metathesis feed stream with ethylene in the presence of the metathesis catalyst and an isomerization catalyst to form the metathesis product stream.

10. The process of claim 9, wherein the isomerization catalyst comprises magnesium oxide.

11. The process of claim 8, wherein ethylene is introduced to the metathesis reaction at a rate sufficient to provide an ethylene:C₄₊ molar ratio contacting the metathesis catalyst of from 1:1 to 2:1.

12. (canceled)

13. The process of claim 8 exhibiting a process efficiency of at least 95%.

14. The process of claim 8, wherein the metathesis catalyst comprises a transition metal oxide.

15. The process of claim 8, wherein the metathesis catalyst comprises tungsten oxide.

16. (canceled)

17. (canceled)

18. (canceled)
